(19) United States
(12) Patent Application Publication
(10) Pub. No.: US 2015/0025256 A1
(43) Pub. Date: Jan. 22, 2015

(54) Purification of 5-Hydroxymethylfurfural (HMF) by Crystallization

(71) Applicants: Tim Ståhlberg, Kobenhavn (DK); Jacob Skibsted Jensen, Herlev (DK); Anders Riisager, Taastrup (DK); John M. Woodley, Kobenhavn Ø (DK)

(72) Inventors: Tim Ståhlberg, Kobenhavn (DK); Jacob Skibsted Jensen, Herlev (DK); Anders Riisager, Taastrup (DK); John M. Woodley, Kobenhavn Ø (DK)

(21) Appl. No.: 14/239,321

(22) PCT Filed: Aug. 17, 2012
(86) PCT No.: PCT/EP2012/066125

§ 371 Date: May 27, 2014

Related U.S. Application Data
(60) Provisional application No. 61/524,963, filed on Aug. 18, 2011.

(30) Foreign Application Priority Data
Aug. 18, 2011 (DK) PA201100627

(51) Int. Cl. C07D 307/48

(52) U.S. Cl. CPC C07D 307/48
USPC 549/488

(57) ABSTRACT

This invention relates to an efficient procedure for purifying HMF by crystallization at low temperature from an organic solvent.

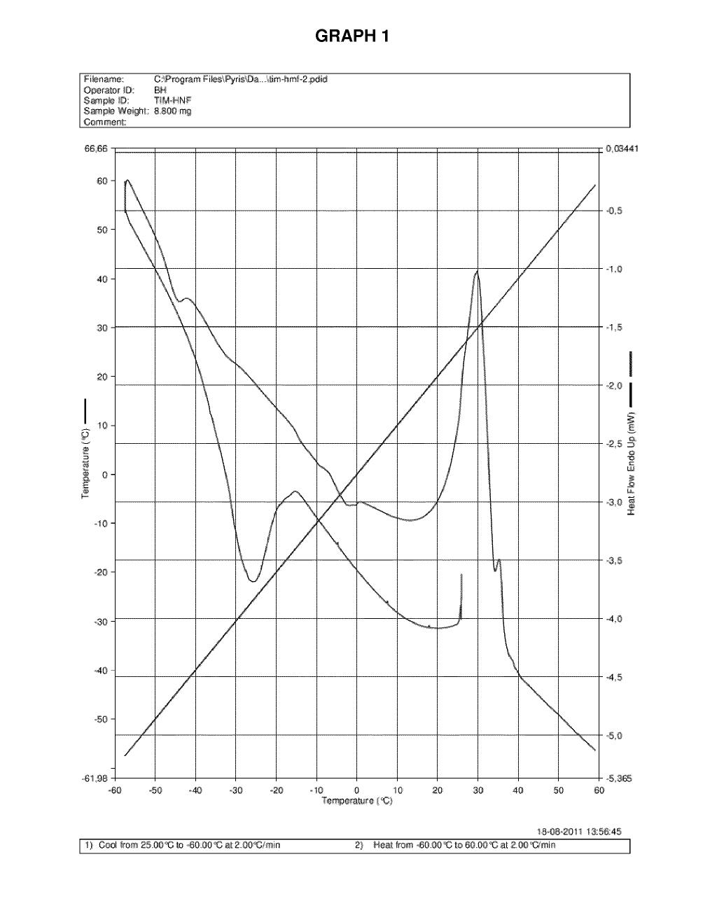

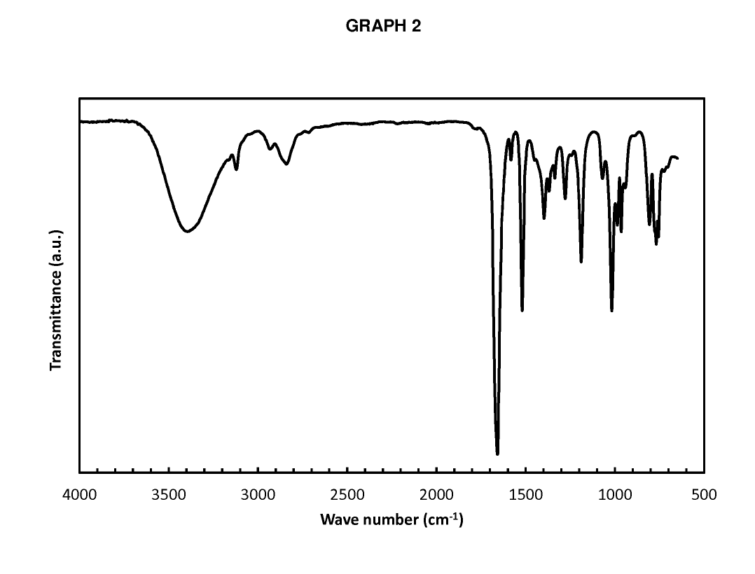

### BACKGROUND

Many chemical compounds needed for various industries have for many years been derived from the petrochemical industry. However, due to increases in the price of crude oil and a general awareness of replacing petrochemicals with renewable resources there has been and still is a wish to base the production of chemical compounds on renewable resources.

5-Hydroxymethylfurfural (HMF) is an example of such a compound because it is derived from dehydration of sugars making it derivable from renewable biomass resources. HMF can for example be converted to 2,5-dimethylfuran which is a liquid biofuel or to 2,5-furandicarboxylic acid by oxidation. The latter compound, 2,5-furandicarboxylic acid (FDA), can be used as a replacement of terephthalic acid in the production of polyesters such as polyethylene-terephthalate (PET) and polybutyleneterephthalate (PBT).

Examples of interesting derivatives produced from HMF in addition to FDA are shown below:

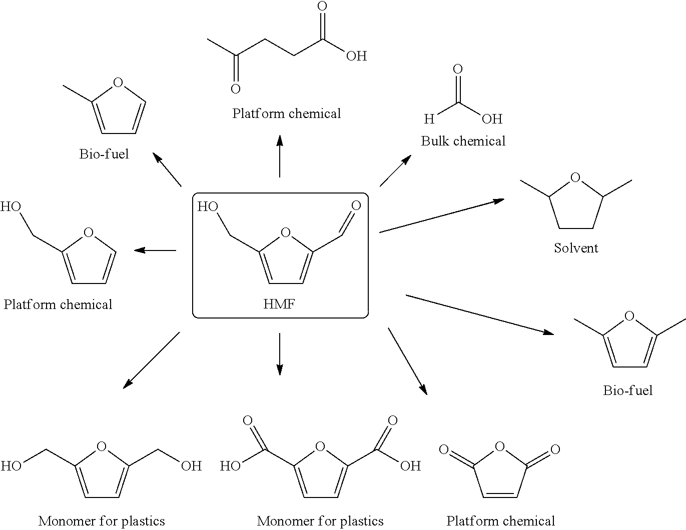

In certain applications the purity of HMF is crucial. For such purposes an efficient purification method that affords HMF with high purity and minimal loss on purification is essential. However, the low melting point of HMF and its high solubility in most common solvents renders its purification by crystallization particularly difficult.

Only a few papers and patents discuss the industrial scale production of HMF, and even fewer discuss the actual isolation of HMF. Furthermore, there is surprisingly little information on the purification of HMF, another vital aspect of large scale production. Even though the exact future application of HMF at this stage is unknown, a robust purification protocol would be of the essence. A few examples can be found in the literature such as purification by distillation, chromatography and crystallization. However, these methods either involve the use of environmentally questionable solvents such as CH₂Cl₂ or excessive solvent use. Moreover, many of the methods have insufficient data on purity. The limited amount of published purification procedures for HMF could be due to its low melting point making crystallization, the preferred purification method for large scale production, more difficult.

There is thus a need for developing an efficient procedure for purifying crude HMF.

### FIGURES

Graph 1: DSC spectrum of HMF recorded on Perkin Elmer PYRIS Diamond DSC comprising an event with a peak at about 30° C. (±2° C.).

Graph 2: FTIR spectrum of HMF recorded on Perkin-Elmer “spectrum one”, 16 scans, 4 cm-1 using Perkin-Elmer “Universal ATR sampling accessory”.

### SUMMARY OF THE INVENTION

In their search for a purification procedure the inventors screened a number of conventional organic solvents in order to attempt a crystallization of crude HMF from a reaction mixture. From this survey it turned out that pure HMF can be precipitated in solid form at temperatures below zero from a solution of crude HMF in an organic solvent or solvent mixture having a freezing point of −50° C. or lower, such as dialkyl ethers R₁—O—R₂, wherein R₁ and R₂ are individually selected from linear C₁-C₆ alkyl groups, branched C₃-C₆ alkyl groups and cyclic C₃-C₆ alkyl groups, which solution may further contain from 0-10% by volume of a different organic solvent selected from esters R₃—COOR₄ and aromatic hydrocarbons ArR₅R₆, wherein R₃ and R₄ are individually selected from linear C₁-C₆ alkyl groups, branched C₃-C₆ alkyl groups and cyclic C₃-C₆ alkyl groups, and wherein ArR₅R₆ denotes a phenyl ring substituted with two substituents R₅ and R₆ individually selected from hydrogen, halogen, linear C₁-C₆ alkyl groups and C₁-C₆ alkoxy groups, and isolated eg. by filtration without melting.

Methyl tert-butyl ether (MTBE) is an example of such a solvent. When decreasing the temperature to −30° C., a white precipitate of HMF was formed from a solution of crude HMF in MTBE. The precipitate could be filtered off and washed with MTBE and subsequently 1-pentane. The low boiling point of pentane enabled efficient drying of HMF without melting the product. The purity was found to be >99% according to HPLC and the yield app 90%.

In a first aspect the invention thus relates to a process for isolating pure 5-hydroxymethyl-furfural (HMF) in solid form, comprising the steps of:
a) Providing a solution of crude HMF in an organic solvent or solvent mixture having a freezing point of −50° C. or lower,
b) Optionally filtering said solution to remove insoluble particles,
c) Cooling the obtained solution to reach a final temperature of between 0 and −40° C.,
d) Optionally adding crystallization seed crystals,
e) Stirring the resulting suspension at the final temperature until the precipitation is deemed complete,
f) Isolating the precipitated solid HMF by filtering the suspension at the final temperature,
g) Optionally washing the isolated solid HMF with a low-boiling hydrocarbon like 1-pentane,
h) Drying the isolated solid HMF, optionally in vacuo.

In a preferred embodiment of the invention the organic solvent or solvent mixture in step a) of the process of the first aspect is selected from one or more dialkylethers R₁—O—R₂ wherein R₁ and R₂ are individually selected from linear C₁-C₆ alkyl groups, branched C₃-C₆ alkyl groups and cyclic C₃-C₆ alkyl groups, which solution further contains from 0-10% by volume of a different organic solvent selected from esters R₃-COOR₄ and aromatic hydrocarbons ArR₅R₆, wherein R₃ and R₄ are individually selected from linear C₁-C₆ alkyl groups, branched C₃-C₆ alkyl groups and cyclic C₃-C₆ alkyl groups, and wherein ArR₅R₆ denotes a phenyl ring substituted with two substituents R₅ and R₆ individually selected from hydrogen, halogen, linear C₁-C₆ alkyl groups and C₁-C₆ alkoxy groups.

In another embodiment the cooling of the solution in step c) is carried out at a rate of between 0.5-2° C. per minute.

In another embodiment the precipitation and isolation of the precipitated solid HMF is conducted at final temperature of between −25 and −35° C., preferably around −30° C.

In another embodiment the solution of crude HMF in an organic solvent or solvent mixture is provided by dissolving crude HMF in 3-5 volumes (L per kg crude HMF) of said organic solvent or solvent mixture, optionally by heating.

In another embodiment the solution of crude HMF in an organic solvent or solvent mixture is provided directly during extractive work-up of the chemical process leading to crude HMF by using said organic solvent or solvent mixture for the extraction, optionally followed by partial removal of said organic solvent or solvent mixture by evaporation until a solution containing 3-5 volumes organic solvent or solvent mixture (L per kg crude HMF) is achieved.

In a preferred embodiment R₁ is methyl and R₂ is selected from tert-butyl and cyclopentyl.

In another preferred embodiment the dialkylether is methyl tert-butyl ether (MTBE).

In another preferred embodiment HMF is isolated in >90% crystalline form having a chemical purity of at least 95% by weight, preferably at least 99% by weight.

In another embodiment the HMF obtainable by a process according to any one of the embodiments of the invention is crystalline and further characterized by having a differential scanning calorimetry curve substantially identical to Graph 1 comprising an event with a peak at about 30° C. (±2° C.).

In another embodiment the HMF obtainable by a process according to any one of the embodiments of the invention is crystalline and further characterized by an ATR-FTIR spectrum substantially identical to Graph 2.

Experimental:

Crude HMF produced from a literature procedure (R. M. Musau et al., Biomass 1987, 13, 67-74) was dissolved in 4 volumes (L solvent/kg product) of MTBE at room temperature. The temperature was decreased to −30° C. and after 30 minutes crystal formation was observed. The mixture was left to crystallize for 12 hours after which the crystals were filtered off at −30° C. The crystals were washed with of 1-pentane (1 volume) and sucked dry (90% yield, >99% pure by HPLC).

The crystals were analyzed by Differential Scanning calorimetry (Graph 1) and attenuated total reflectance Fourier transform infrared (ATR-FTIR) spectroscopy (Graph 2).

The described procedure is superior compared to previously described crystallization methods because of the high yield (>90%) and high purity (>99%) of the isolated product which may be achieved, and because industrially acceptable solvents like MTBE may be employed. Furthermore, the described procedure is superior to chromatographic methods because of the modest consumption of solvent. Finally, the overall procedure may be further optimized using appropriate anti-solvents, by applying seeding and/or by adjusting temperature ramps during the precipitation step.

1. A process for isolating pure 5-hydroxymethylfurfural (HMF) in solid form, comprising the steps of:
a) Providing a solution of crude HMF in an organic solvent or solvent mixture having a freezing point of −50° C. or lower,
b) Optionally filtering said solution to remove insoluble particles,
c) Cooling the obtained solution to reach a final temperature of between 0 and −40° C.,
d) Optionally adding crystallization seed crystals,
e) Stirring the resulting suspension at the final temperature until the precipitation is deemed complete,
f) Isolating the precipitated solid HMF by filtering the suspension at the final temperature,
g) Optionally washing the isolated solid HMF with a low-boiling hydrocarbon like 1-pentane,
h) Drying the isolated solid HMF, optionally in vacuo.

2. The process of claim 1 wherein the organic solvent or solvent mixture in step a) is selected from one or more dialkylethers R₁—O—R₂ wherein R₁ and R₂ are individually selected from linear C₁-C₆ alkyl groups, branched C₃-C₆ alkyl groups and cyclic C₃-C₆ alkyl groups, which solution further contains from 0-10% by volume of a different organic solvent selected from esters R₃—COOR₄ and aromatic hydrocarbons ArR₅R₆, wherein R₃ and R₄ are individually selected from linear C₁-C₆ alkyl groups, branched C₃-C₆ alkyl groups and cyclic C₃-C₆ alkyl groups, and wherein ArR₅R₆ denotes a phenyl ring substituted with two substituents R₅ and R₆ individually selected from hydrogen, halogen, linear C₁-C₆ alkyl groups and C₁-C₆ alkoxy groups.

3. The process of claim 1 wherein the cooling of the solution in step c) is carried out at a rate of between 0.5-2° C. per minute.

4. The process of claim 1, wherein the precipitation and isolation of the precipitated solid HMF is conducted at final temperature of between −25 and −35° C., preferably around −30° C.

5. The process of claim 1, wherein the solution of crude HMF in an organic solvent or solvent mixture is provided by dissolving crude HMF in 3-5 volumes (L per kg crude HMF) of said organic solvent or solvent mixture, optionally by heating.

6. The process of claim 1, wherein the solution of crude HMF in an organic solvent or solvent mixture is provided directly during extractive work-up of the chemical process leading to crude HMF by using said organic solvent or solvent mixture for the extraction, optionally followed by partial removal of said organic solvent or solvent mixture by evaporation until a solution containing 3-5 volumes organic solvent or solvent mixture (L per kg crude HMF) is achieved.

7. The process of claim 1, wherein R₁ is methyl and R₂ is selected from tert-butyl and cyclopentyl.

8. The process of claim 1, wherein the dialkylether is methyl tert-butyl ether (MTBE).

9. The process of claim 1, wherein HMF is isolated in >90% crystalline form having a chemical purity of at least 95%by weight.

10. Crystalline HMF obtainable by a process according to claim 1 further characterized by having a differential scanning calorimetry curve substantially identical to Graph 1 comprising an event with a peak at about 30° C. (±2° C.).

11. Crystalline HMF obtainable by a process according to claim 1 further characterized by an ATR-FTIR spectrum substantially identical to Graph 2.

12. The process of claim 1, wherein HMF is isolated in >90% crystalline form having chemical purity of at least 99% by weight.
